(19) United States
(12) Patent Application Publication
(10) Pub. No.: US 2012/0040864 A1
(43) Pub. Date: Feb. 16, 2012

(54) Method for Determining the Cbl-b Expression

(75) Inventors: Hans Loibner, Vienna (AT); Gottfried Baier, Innsbruck (AT); Gunther Lametschwandtner, Vienna (AT); Manfred Schuster, Schrick (AT); Thomas Gruber, Brixen i. T. (AT); Dominik Wolf, Mutters (AT)

(73) Assignees: MEDIZINISCHE UNIVERSITAET INNSBRUCK, Innsbruck (AT); APEIRON BIOLOGICS AG, Vienna (AT)

(21) Appl. No.: 13/264,463

(22) PCT Filed: Apr. 14, 2010
(86) PCT No.: PCT/EP2010/054886

§ 371 Date: Oct. 14, 2011

(30) Foreign Application Priority Data
Apr. 14, 2009 (AT) A 573/2009

(51) Int. Cl. G01N 21/64; G01N 33/566; C40B 30/04; C07K 16/40

(52) U.S. Cl. 506/9; 530/389.6; 435/7.1

(57) ABSTRACT

The present invention relates to methods of determining intracellular Cbl-b protein in cells of a sample, comprising
introducing an antibody, which binds Cbl-b intracellularly, into a cell,
allowing contracting of the antibody and Cbl-b protein potentially present in the cell,
detecting binding events between the antibody and Cbl-b,
quantifying the detected binding events,
whereby the content of Cbl-b protein is determined.

The present invention relates to methods of determining intracellular proteins and biomarkers.

The outcome of a number of life-threatening diseases essentially depends on the reactions of the patient's own immune system. This very clearly applies in the case of infectious diseases, and is of particular relevance in chronic infectious diseases in which persistent establishment of the pathogen in the patient's body can occur. One of the mechanisms responsible for this is the formation of so-called regulatory T-cells, which subsequently suppress the immune response of effector cells against the pathogen. This suppression of effector T-cells takes place, among other things, through adenosine which is generated by regulatory T-cells and can transform T-cells into an anergic state. T-cells which are in such an anergic state have an increased intracellular content of the E3-ubiquitin ligase Cbl-b.

In the absence of Cbl-b, administered, but hardly immunogenic substances, can induce a strong immune response. In addition, Cb1-1 deficient mice (homozytocic gene knock-out) are viable and their immune system is able to efficiently recognise autologously-induced tumours and to build up a lytic immune response mainly based on CD8+ T-cells (Loeser et al., JEM (2007) doi:10.1084/iem.20061699). However, the described complete elimination of the enzyme also led to increased autoimmunity after immunisation with superantigens. Loeser at al. could demonstrate that Cbl-b as a negative regulator is essentially responsible for the “immune reactivity” of T-cells.

The determination of the intracellular Cbl-b protein in the patient's T-cells is therefore a relevant biomarker for the status of the immune response to certain antigens. This enzyme constitutes a switching point in steering the immune reactivity (Chiang et al., J Clin Invest (2007) doi:10.1172/JCI29472).

Zhou et al. (Neurosci. Lett. (2008), doi: 10.1016/j.neulet.2008.05.089) demonstrate a link between the quantity of Cbl-b, measure by way of western blotting of cell homogenisates, and multiple sclerosis. The drawback of this method is that the evaluation of cell homogenisates of various cells does not permit simple differentiation between active and inactive immune cells.

Leng et al. (Int. Immunol. (2006) 18(5): 637-44) describe a study of TGF-beta, Cbl-b and CTLA-4 values in various stages of immune activation. Cbl-b was detected through antibodies in cell lysates by way of western blotting.

Babu et al. (J. Immunol. (2006) 176(5): 3248-56) describe Cbl-b-induced anergy in immune cells. Cbl-b assays were based on quantitative RT-PCR.

WO 2004/108896 A2 relates to gene expression profiling in uterine and ovarian cancer. The Clb-b gene is also among the studied genes.

WO 2008/021431 A2 relates to the monitoring of organ transplantations and immune disorders, whereby the Clb-b gene was monitored.

It is therefore one of the aims of the invention to be able to determine clinically relevant Cbl-b quantities in cells.

The present invention relates to a method of determining intracellular Cbl-b proteins in cells of a sample, comprising
introducing an antibody, which binds Cbl-b intracellularly, into a cell,
allowing contacting of the antibody and Cbl-b potentially present in the cell,
detecting binding events between the antibody and the Cbl-b, and, if necessary
quantifying of the detected binding events, whereby the content of Cbl-b protein is determined.

The present invention therefore relates to the direct measurement of the intracellular content of Cbl-b in immune cells, which, for example, can be obtained directly from the blood or other tissues (e.g. tumour tissue, organ biopsies, intestinal biopsies as well as lavage, joint fluid, cerebrospinal fluid etc.) of patients. For further analysis the patient's cells can by way of in vitro or ex vivo methods be brought into contact with an antigen, which, for example, is functionally related with a relevant disease (e.g. a pathogen isolated for an infectious disease, tumour antigens in the case of a cancerous disease, autoantigens in autoimmune diseases, alloantigens in allotransplants, allergens in the case of allergies etc.) in order to determine the immune reactivity of the cells to such stimulants.

The genetic products of the Cbl-b gene are described in detail in the prior art (UniGene Id. Hs.3144 and Hs.381921). Cbl-b sequences are, for example, published in the NCBI GenBank database under acc. no. DQ349203 (nucleic acid) and ABC86700 (protein). Anti-Cbl-b antibodies are commercially available though none of them have so far been designated for the determination of intracellular Cbl-b protein content.

The intracellular measurement of certain proteins through antibodies depends on various factors which are not comparable with batch methods, such as, for example, the measurement in homogenisates for western blots. On the one hand, in intracellular measurement an antibody has to be introduced into a cell. For this the cell is made permeable as a result of which certain molecules can penetrate into the cell through artificially created pores. This penetration is not possible in the case of all antibody sizes. The antibodies should be kept as small as possible. For intracellular measurement antibodies can be modified in order to apply a marker. Normally fluorescence stains are used as markers in intracellular measurement. In this way in current methods a problem can arise with a lower detection limit and an increased signal/noise ratio.

A further factor that has to be taken into account in intracellular measurement is that cell components should not diffuse out of the cell during permeabilisation. The cells are therefore fixed. This means fixing at least for the measurement of relevant cell components (proteins, ions and small molecules can diffuse out). For this proteins, and possibly also nucleic acids are cross-linked by means of cross-linking reagents so that they form a stable network frame. An example of such a cross-linking reagent is formaldehyde. So that an antibody is suitable for the intracellular determination of Cbl-b, it must preferably be able to recognise its cross-linked form. In a cells proteins are not, or only to a small degree, present in isolated form, but form complexes with various binding partners. In particular, phosphoepitopes, which also occur on Cbl-b, are generally concealed through the formation of complexes with other proteins Krutzik et al., Clin. Immun. 110 (2004): 206-221).

Antibodies suitable for intacellular measurement should be able to recognise the protein in its three-dimensionally folded state. As many antibodies that have been generated with the aid of peptides or short recombinant fragments of the antigen can preferably recognise linear epitopes, this does not directly result in suitability for intracellular applications. The intracellular fixation of the cell often also makes recognition of epitopes by antibodies more difficult. The antibodies may well recognise denatured Cbl-b, but only in the form of linear epitopes and no longer in the cellular context of the complete protein in permeabilised and fixed cells.

Surprisingly it could be shown in accordance with the invention that at least one antibody is still suitable for determining the Clb-b protein content in cells even after cell fixation.

The detection of the binding events between the antibody and Cbl-b can take place in a conventional manner, such as by way of labelling the antibodies, whereby only those antibodies are detected which also bind Cbl-b proteins in the cells. Unbound antibodies could be removed by means of a washing stage. An appropriate form of labelling is, for example, fluorescence labelling or radioactive labelling. Enzymatic labelling should not be technically ruled out, but may not be suitable for certain applications and cell permeabilisation methods.

Detection itself, can take place, for example, using suitable detectors, whereby signals may also be amplified by photomultipliers. Suitable detection means include a light source suitable for the fluorescence stimulation of a selected fluorescence label and an optical detector. Detection of the cells can, if necessary, take place in a measuring cell, such as a throughflow cell, in which the cells are passed through from, for example, a cell suspension.

The term “antibody” in accordance with the present invention relates to all types of antibody and functional antibody equivalents, more particularly antibodies of type IgA, IgD, IgE, IgG, IgM including all sub-types such as IgG1 or IgG2, as well as functional antigen-specific fragments such as Fab, F(ab)₂, Fv etc. Equally, artificial and artificially modified antibodies, such as, for example, single chain antibody fragments (scFv) are understood as “antibodies” in the present invention.

The antibody can be monoclonal or polyclonal. It can originate from any organism (including isolated cells therefrom), more particularly a mammal, specifically a primate or a human, or a rodent such as a mouse, rat or hamster.

In preferred forms of embodiment the antibodies are labelled, preferably fluorescence-labelled.

In the further forms of embodiment the cells include leukocytes, preferably PBMCs (mononuclear peripheral blood cells). In preferred forms of embodiment the cells to be used in accordance with the invention are leukocytes (T-lymphocytes, B-lymphocytes, NK cells or NKT cells, monocytes, macrophages and/or dendritic cells) more particularly PBMCs, T-lymphocytes, CD8+ T-lymphocytes, CD4+ T-lymphocytes, especially Th1, Th2, Th17, Tregs (regulatory T-cell). The differentiation of the various T-cell sub-populations can include surface markers, preferably CD4, CD8, CD25, CD69, CD70, CD27, CD39, CD54, CD45RA, CD45RO, CD62L, CD73, CD95, CD107a, CD127, CD134, CDw137, CD152, CD154, CCR4, CCR6, CCR7, CCR8, CXCR3, GITR, PD-1, A2AR, cytokines, more particularly IL-2, IL-6, IL-7, IL-10, IL-15, IL-17A, IL-17F, IL-21, IL-22, IL-26, IL-27, interferon-g, lymphotoxin-a, TNF-a, and other intracellular molecules, more particularly Foxp3, GATA-3, RORc, T-bet. The differentiation of the various sub-populations of NK cells is also possible, preferably on the basis of expression of CD1, CD3, CD16, CD69, CD95, CD107a, CD127, KIR- and NKR-molecules. In addition the differentiation of various B-cell sub-populations is possible, preferably on the basis of the expression of CD19, CD20, CD22, CD27, CD38, CD40, CD267, CD268, CD269, (membrane-bound) IgD.

In addition, the reactivity of leukocytes of individuals to certain antigens in various sub-fractions of immune cells can be determined. For this the leukocytes are isolated from blood or tissue and then brought into contact with the relevant antigen for the disease in question. This can take place through direct addition to the unseparated leukocyte preparation (e.g. PBMCs). Contacting with the antigen can also take place in vivo—e.g. during the course of an illness. Alternatively antigen-presenting cells, preferably dentritic cells, monocytes, macrophages or B-cells, can be used for the presentation of the antigen. Lymphocytes, preferably T-cells, can then be brought into contact with such antigen-loaded cells in order to achieve an antigen-specific in vitro stimulation. The T-cells stimulated in this way can then be examined for Cbl-b expression after a certain period of time, preferably after 4, 8, 12, 16, 24, 36, 48, 72, 96, 120, 144, 168, 192, 216, 240 hours, and the Cbl-b correlation can be correlated with the expression of the previously listed molecule classes.

Preferably the cells are measured individually for detection of the binding events, preferably with simultaneous classification or determination of the cell type. Through individual measurement of the cells it is possible to isolate those cells from a cell population which exhibit a particularly high or particularly low quantity of Cbl-b protein. In the methods used to date in which, for example, entire cells fractions were opened up, there is always the risk of only a mean value being determined, with particularly activated or inactivated cells no longer being identified by the Cbl-b content. If particularly high Cbl-b quantities and low Cbl-b quantities are simultaneously present in other cells, only a mean value would be determined which does not allow any conclusions to be drawn about any special immunological behaviour. With the individual measurement of cells it is possible to simultaneously use, for example, different markers, more particularly differently coloured fluorescence markers, which provide a second signal on the basis of determined cell surface markers with which cell types can be differentiated as has already been set out above.

In special cases, with the method in accordance with the invention the cells with a high throughput of at least 20, preferably at least 50, more particularly at least 100 and especially preferably 200, cells per second are measured. High throughput methods have the advantage that a large number of cells can be measured per unit of time, whereby it is also of advantage if one or more further markers beside Cbl-b can be measured at the same time and parallel allocation or sorting of the cells in accordance with the criteria is made possible. One such method is, for example, flow cytometry, with which up to 1000 cells per second or more can be categorised and measured by Cbl-b content. Preferably fluorescence dyes are used for the detection of Cbl-b and other cellular markers (“multicolour”-based method). Through the additional measurement of other intracellular proteins it is also possible to standardise and collate the Cbl-b quantity, if, in addition to the Cbl-b quantity other control values or control proteins are measured which represent a constant reference value for the cells of interest and are suitable for the standardisation and comparison of the Cbl-b values.

The determination methods (western blot) used in the literature do not differentiate between the various fractions of cells or cell types. Such differentiation would only be possible if the intracellular content of Cbl-b in the corresponding sub-fractions of cells could be itemised in detail. In principle such an analysis is possible by means of multicolour-based flow cytometry methods. However, so far it has not been possible to establish such a method suitable for clinical applications. This was made possible for the first time with the present invention through the provision of a practical method of determining the Cbl-b protein content in various sub-fractions of cells of the immune system, more particularly T-cells.

In accordance with the invention, in the quantification of Cbl-b, it is also possible to differentiate the Cbl-b quantity in the individual cells or also to quantify the cells in which Cbl-b is detected (as of a certain threshold value). In one embodiment of the method in accordance with the invention the proportion of cells in which Cbl-b is detected and/or the quantify of Cbl-b protein in the cells is also quantified.

By way of the determination of the content of Cbl-b protein in the cells it is also possible to assess the immune reactivity of the cells to certain immunological events, such as, for example, exposure to an antigen. For this reason in a further embodiment the cell is stimulated with an antigen before detection of the binding events, preferably also before introduction of the antibody, whereby preferably the cells include antigen-presenting cells. If the cells are then stimulated through contact with the antigen, this then leads to a considerably increased quantity of the Cbl-b protein if anergy sets in, in contrast to optimum stimulation of the cells. The extent of cell stimulation can also be determined through simultaneously measuring further markers and in this way a Cbl-b increase through cell stimulation can be differentiated from the Cbl-b increase through anergy.

In an analogue manner to antigens, cells can also be treated with further immunomodulating substances, such as cytokines or ligands of immunomodulting receptors. Therefore, the cells are preferably treated during or before detection of the binding events with immunostimulating substances, preferably cytokine(s) or ligands of immunomodulating receptors, more particularly TLR (toll-like receptors) or antibodies to surface molecules, more particularly CD3 and/or CD28.

Cbl-b is a potentially phosphorylated or ubiquitinated protein. Through the selection of suitable antibodies or suppression of the detection of binding events with Cbl-p without phosphate or ubiquitin residue selections of the detected Cbl-b can be carried out. Therefore in specially preferred embodiments the quantity of postranslationally modified, preferably phosphorylated and/or ubiquitinated Cbl-b protein is determined.

A further aspect of the present invention relates to a method of diagnosing a disease or predicting the occurrence or course of a disease, comprising
determination of the Cbl-b protein content in cells of a subject as described herein, preferably on two different days,
comparison of the Cbl-b protein content of cells of diseased or healthy reference subjects,
determination of a difference between the Cbl-b protein content of the subject and the reference subjects, whereby a disease or the prognosis is determined.

The present invention describes for the first time a method of, for example, flow cytometric determination of the Cbl-b protein content in leukocytes and thereby allows a detailed analysis of the immune status of the patient. For determining the Cbl-b protein content in the patient's leukocytes, the latter are isolated from patient tissue, preferably peripheral blood, bodily fluids or tissue biopsies.

In order to monitor the course of a disease or to predict the occurrence of a disease in terms of the change in the intracellular content of Cbl-b, measurements of the Cbl-b protein content are carried out at 2, preferably 3, particularly preferably 4 or more different times. These data can then be correlated with the Cbl-b protein content of the reference subject in order to identify significant deviations from a healthy state or characteristic of the course or occurrence of certain diseases. These different times can be at intervals of at least 4, at least 8, at least 12, at least 16, at least 24, at least 36, at least 48, at least 72, at least 96, at least 120, at least 144, at least 168, at least 192, at least 216 or at least 240 hours, at least 2 days, preferably at least 1 week, particularly preferably at least 2 weeks or 1 month or more.

Preferably the subject is a mammal or a bird, preferably a primate, human, rodent, more particularly a mouse, a rat, a rat, a domestic animal, more particularly a pig, horse, cow, chicken, turkey, dog or cat. Particularly preferably the subject is a human.

As has already been set out above, before determining the content of Cbl-b protein in the cells, these cells can be brought into contact with a certain antigen in order to determine a particular immunological reaction. Preferably an antigen is selected which is linked to the diseases, for example, which can trigger or influence the diseases. Such antigens are, for example, allergens or immunogens of pathogens. This also includes the use of epitopes of the antigens. Cancer antigens or cancer epitopes can also be selected.

Preferably, in the diagnosis and/or prognosis, measurement of other cells markers is carried out, more particularly to differentiate certain cell types and populations. For certain diseases a particular cell type and/or a particular cell population is decisive (or causal) and the relevant cell group can be specifically addressed in the diagnosis or prognosis.

The diseases which can be examined in accordance with the invention are all those associated with influencing an immunological response. More particularly diseases in which a change in the immune response is the cause of the disease are preferred. The term “disease” should be understood as a general condition harmful to health, which differs from a normal state of a healthy person.

A particularly special disease is chronic infection. In accordance with the invention, via Cbl-b as the biomarker, it can be determined whether an immune response to a certain infection (e.g. bringing immune system cells into contact with an antigen as described above) is sufficient to fight an infection or whether there is a risk of a chronic infection developing from an infection which cannot adequately or successfully be prevented by the immune system.

A further disease which can be determined or predicted in accordance with the invention is a tumor disease. Tumours which are not adequately fought by the subject's immune system are able to persist and/or spread. It could be shown that Cbl-b is a jointly responsible immunomodulator, which in upregulation or at least non-downregulation leads to tumours not being adequately fought by the immune system with certain tumour antigens. Thus tumorous diseases are a further important area of application for Cbl-b as a biomarker. In many tumorous diseases the proportion of regulatory and anergic cells in the tumour environment is seen as a negative prognostic marker. Therefore here too the determination of the protein content of Cbl-b in the immune cells both in the tumour and circulating (more particularly in T-cells and NK cells) is relevant biomarker. As a certain proportion of T-cells found in a certain tissue (“homing”) also circulates through the blood, the determination of Cbl-b in the immune cells of the peripheral blood of the patient can also be used as a biomarker.

In other embodiments the disease is an inflammatory or autoimmune disease. Other areas of application for Cbl-b as a biomarker are autoimmune diseases (e.g. MS, colitis, psoriasis, arthritis, SLE) as well as inflammatory diseases (e.g. allergic asthma). The occurrence of these immune disease is causally related to the reaction to endogenous antigens or harmless exogenous antigens. Such an autoimmune reaction or allergic immune reaction to harmless exogenous antigens is normally suppressed by regulatory T-cells, and T-cells which also exhibit a certain reactivity to endogenous antigens are therefore in an anergic state. However, during the course of (auto) immune disease autoreactive T-cells are activated and chronic inflammatory processes develop in affected tissue. It has already been shown that the quantity of Cbl-b in peripheral lymphocytes of the blood differed significantly between multiple sclerosis(MS) patients and healthy reference persons (Zhou et al., Neuroscience Letters 2008 Aug. 8; 440(3):336-9). In addition there was a highly significant correlation with the current state of the MS patients (relapse versus remission).

In certain embodiments the disease includes/is an immune reaction for allotransplantates. Using Cbl-b as a biomarker the monitoring of transplantate rejection in patients with allotransplantates is possible. Here too there is a need for biomarkers which can be determined without a biopsy of the transplanted organ. As in the immune tolerance to the transplantate the same molecular mechanisms as set out about are relevant, Cbl-b expression in leukocytes is also a suitable biomarker for the immune status of patients in relation to rejection of the transplanted organ.

In particular in special embodiments the disease can include an immune reaction to allergens, exogenous antigens or endogenous antigens (autoreactivity). Allergies are among the classic immunomodulated diseases which can, for example, also be decisively influenced by downregulation of Cbl-b. In this way it is possible to use Cbl-b as a marker for the diagnosis or prognosis of the course of the disease.

Another area of application of Cbl-b as a biomarker is the determination of the general disposition of still healthy individuals to immunological reactivity. As this disposition influences the individual reaction to both exogenous and endogenous antigens, its determination is of relevance for predicting the reaction of healthy individuals to antigens introduced into the body through vaccination, infection or other contact as well as the disposition with regard to immunological autoreactivity. A further aspect of the present invention therefore relates to a method of determining the immune reactivity of cells of a subject, more particularly leukocytes, to an antigen comprising
bringing the cells into contact with the antigen,
determining the Cbl-b protein content in the cells of a sample of the subject are described herein,
comparing the Cbl-b protein content with reference values of a Cbl-b protein content in the case of immune reactivity to a reference antigen or absence of an immune reactivity to a reference antigen,
determination of a difference between the Cbl-b protein content of the subject and the reference values.

Therefore Cbl-b expression can be used a biomarker for the immunological disposition of individuals in terms of reactivity to allergens, exogenous or endogenous antigens (autoreactivity).

The expression of Cbl-b in T-cells is, among other things, dependent of the activation state of the cells. T-cell activation leads to an increase in the quantity of Cbl-b mRNA and protein. This means that it is not clear from the start whether changes in the total quantity of Cbl-b in leukocytes of the peripheral blood are due to full functional T-cell activation itself or to an anergic phenotype, whereby it is of advantage to also distinguish whether the cells promote an immune reaction (T_{H}, T_{C}) or throttle it (T_{reg}). In the peripheral blood T-cells do not only contain Cbl-b protein but almost all sub-types of leukocytes. It is therefore advantageous to be able to specifically determine the quantity of Cbl-b protein only in certain fractions of T-cells. In accordance with the invention this is made possible, for example, through co-determination of the cell type, at least to differentiate immune response-intensifying or weakening cells.

The reference values for determining a significantly different Cbl-b quantity can be determined from samples from other subjects, preferably with the antigen with which the cells are brought into contact being identical to the reference antigens, in order to match/normalise the general reactivity of the antigen with cells. Some antigens tend toward strong, and others to weak binding and cell activation. Preferably in this method too the antigens are allergens, exogenous antigens or endogenous antigens of the subject.

In the determination of the immune reactivity of cells of a subject the above parameters or selection of the cells (e.g. Kobe determination of special classification markers) are also implemented.

Described herein is the selection of an antibody which is suitable for the intracellular binding of Cbl-b, more particularly which binds an epitope of Cbl-b in the intracellular environment especially after fixation, in particular cross-linking in the cellular context. Such an antibody is also a subject matter of the invention, more particularly for use in the intracellular determination of Cbl-b. Thus the present invention provides as a further aspect the use of an antibody which binds Cbl-b intracellularly for the intracellular determination of Cbl-b. Also included are antibody derivatives or fragments, as already described herein. The antibody is preferably directed against the C-terminal Cbl-b (or is specific to this). In special forms of embodiment the antibody binds an epitope in the area of the C-terminal 300, preferably 250 or 200, preferably 180, particularly preferably 170, particularly preferred 150 or 149, amino acids of Cbl-b. Preferably is specific or directed to the amino acids from 833 to the C-terminal, preferably amino acids 833 to 964 of Cbl-b (or binds an epitope in this range), whereby the numbering of the amino acids corresponds to human Cbl-b. The antibody can be produced, for example, through immunisation with a fragment containing amino acids 833-964 of Cbl-b. The antibody can be from any organism, more particularly mammals and rodents as set out above. An example of an antibody which can be used in accordance with the invention is the antibody abcam ab54362 (commercially available from Abcam, www.abcam.com/CBLB-antibody-246C5a-ab54362.html), a monoclonal murine antibody produced against a recombinant C-terminal fragment (aa833-964) of human Cbl-b. Such an antibody can be used in a method in accordance with the invention. More particularly the antibody is used for determining a disease as described herein.

A further aspect of the invention relates to a kit comprising an antibody, preferably marked, more particularly fluorescence marked, and cell fixation means and/or cell permeabilisation means, preferably selected from formaldehyde, methanol, ethanol, acetone, triton X-100 (octoxynol-9) and saponin, preferably also one or more antibodies to surface receptor of lymphocytes, preferably selected from CD3, CD4, CD8, CD19, CD25, CD45RA, CD45RO, CD69, or also CD4, CD8, CD25, CD69, CD70, CD27, CD39, CD54, CD45RA, CD45RO, CD62L, CD73, CD95, CD107a, CD127, CD134, CDw137, CD152, CD154, CCR4, CCR6, CCR7, CCR8, CXCR3, GITR, PD-1, A2AR, cytokines more particularly IL-2, IL-6, IL-7, IL-10, IL-15, IL-17A, IL-17F, IL-21, IL-22, IL-26, IL-27, interferon-g, lymphotoxin-a, TNF-a, and other intracellular molecules, more particularly Foxp3, GATA-3, RORc, T-bet, and other surface markers for the functional characterisation of immune cells other than CD4 or CD8 T-cells such as CD1, CD3, CD16, CD69, CD95, CD107a, CD127, KIR- and NKR-molecules, CD19, CD20, CD22, CD27, CD38, CD40, CD267, CD268, CD269, IgD.

The present invention is illustrated by way of the following figures and examples without being restricted thereto.

### FIGURES

FIG. 1 shows that in human T-cells the Cbl-b protein content is much higher though the anergy-mediated sole stimulation of the T-cell receptor than that of optimally stimulated (anti-CD3 and anti-CD28) T cells, and high Cbl-b expression can thus be used as a marker of anergic T-cells.

FIG. 2 shows the testing of various antibodies directed against human and murine Cbl-b, as to whether they are suitable for determining the Cbl-b protein content in fixated and permeabilised murine leukocytes in the flow cytometric determination method.

FIG. 3 shows the correlation of the expression determination of Cbl-b by way of RT-PCR (A), western blot (B) and icFACS (C) of human T-cells and thus the validation of the Cbl-b specificity of the icFACS staining of Cbl-b by specific silencing of Cbl-b expression through siRNA directed against Cbl-b.

FIG. 4 shows the simultaneous FACS determination of the Cbl-b protein content of human immune cells from peripheral blood (PBMCs) and the expression of two further immune cell markers (CD45RA and CD3). A: Cbl-b and CD3; B: CD45RA and Cbl-b; C: CD45RA and Cbl-b of the CD3-negative cells; D: Cbl-b expression in CD14-positive and negative myeloid cells

FIG. 5 shows the FACS determination of Cbl-b expression together with CD45RA in NK-cells.

FIG. 6 shows that patients suffering from an autoimmune disease exhibit a reduced Cbl-b content in their T-cells, which also cannot essentially be induced through normally anergy-triggering antigen contact. A: Comparison of the proportion of cells with a low Cbl-b content in the lymphocytes of SLE patients and health reference subjects; B: Cbl-b content CD3+ cells of SLE patients and healthy reference subjects; C: anergic Cbl-b stimulation of SLE patients and healthy reference subjects through an allergen.

### EXAMPLES

### Example 1

### Anergic T-Cells have a Particularly High Content of Intracellular Cbl-b Protein

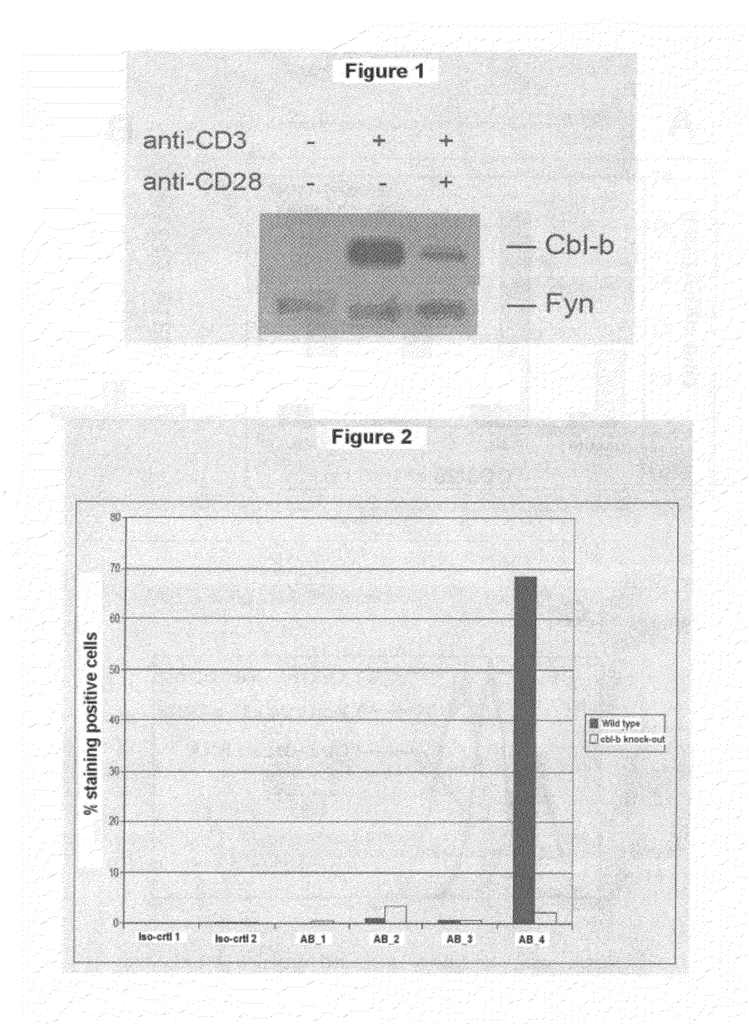

For FIG. 1 PBMCs of healthy volunteer donors were prepared by means of the standard protocol of density gradient centrifuging (Ficoll) and the CD8 T-cells isolated by MACS (Miltenyi, protocol in accordance with the manufacturer's recommendations). The T-cells were then stimulated by means of anti-CD3 or anti-CD3 and anti-CD28 antibodies, re-harvested after 24 hours, and the quantity of Cbl-b protein was determined by means of western blotting using anti-Cbl-b antibodies. This shows that a particularly high Cbl-b protein content is achieved through the anergy-mediating sole stimulation of the T-cell receptor.

### Example 2

### Determination of the Intracellular Content of Cbl-b in Primary Murine Splenocytes by Means of Flow Cytometry

For establishing a protocol for staining with specific antibodies for subsequent determination by means of flow cytometry it is important to validate the specificity of the staining Ideally for checking the specificity cells are used which no longer contain the protein to be determined. Unfortunately there are no human cells available which have been made fully genetically deficient of Cbl-b, but only murine cells from Cbl-b knock-out mice. However the homology of human and murine Cbl-b protein is extremely high (>=95%). This is also reflected in the specification of the tested anti-Cbl-b antibodies described as reactive both to human and murine Clb-b, though only for applications other than flow cytometry. None of the tested antibodies was described as functional in flow cytometry. However, a test of a panel of commercially available antibodies produced the surprising result that one of the tested antibodies could after all specifically stain wild-type cells, though not cells of Cbl-b deficient mice. This antibody (antibody 4) is the antibody Abcam ab54362, produced against a recombinant C-terminal fragment (aa833-964) of human Cbl-b. FIG. 2 shows a summary of these test series, showing the percentage of cells lying in the positive marker region in the histogram of the flow cytometrically-detected antibody-mediated fluorescence.

The cells were stained in accordance with the following protocol:

one million cells were washed once with 200 μl FACS buffer (PBS+2% FCS) and then fixated and permeabilised through incubation for 20 minutes in 250 μl Cytofix/Cytoperm solution (manufacturer: Becton Dickinson). The cells were then washed once with 200 μl Perm/Wash puffer by the same manufacturer and incubated with antibodies (diluted in Perm/Wash buffer to an antibody concentration of 2 μg/ml) at room temperature for 30 minutes. The cell were then washed twice in 200 μl Perm/Wash buffer and incubated with a fluorescence-labelled secondary antibody (anti-mouse IgE-PE, manufacturer Southern Biotech) for a further 30 minutes. Finally the cells were washed once with Wash/Perm buffer and once with FACS butter and re-suspended in 250 μl FACS buffer for the FACS analysis.

As the fluorescence stain of the secondary antibody can be freely selected in this protocol, all possible multicolour stains with other markers can take place in order to specifically detect the Cbl-b expression in certain sub-populations of cells.

### Example 3

### Validation of the Intracellular Cbl-b Staining Protocol Though the Inhibition of Cbl-b Expression Through cblb-Specific siRNA Prior to Cbl-b Determination

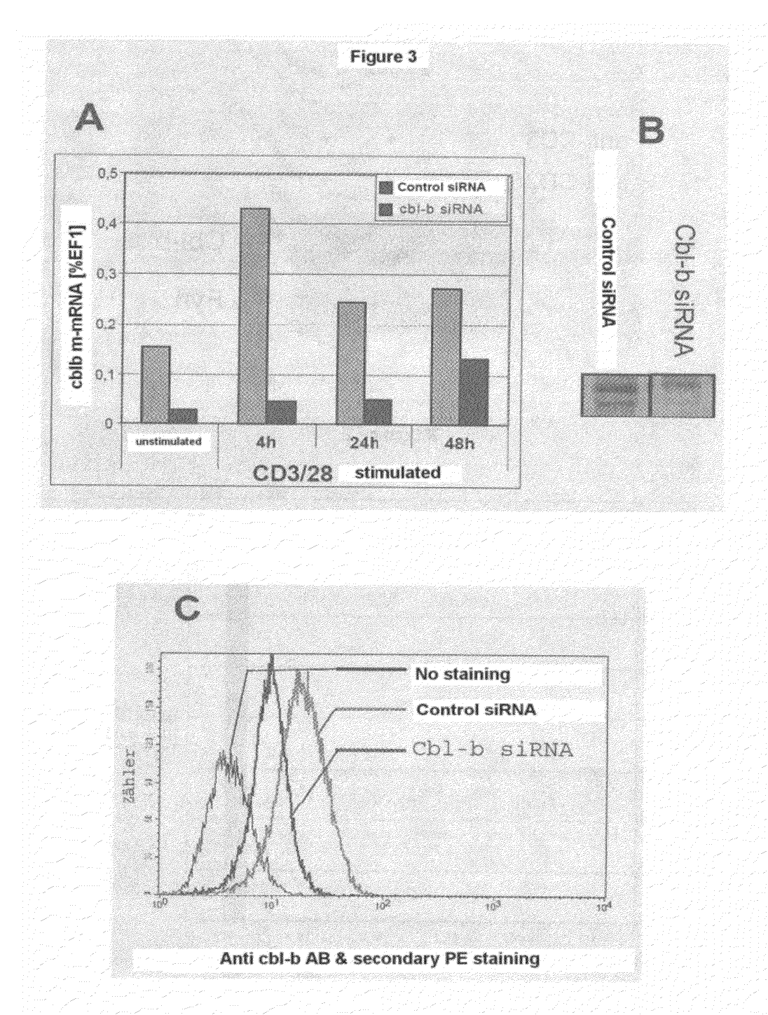

In order to show that detection through the above-described anti-Cbl-b antibody is specific for Clb-b, human T-cells were isolated as described in example 1 and transfected with Clb-b siRNA by way of nucleofection. The inhibition of mRNA resynthesis of Clb-b was confirmed by means of quantitative real-time PCR (FIG. 2A). Consequently a significant reduction in Cbl-b was seen in the western blot after 24 hours anti-CD3/28 stimulation (FIG. 3B).

To stain intracellular Cbl-b the human T-cells were treated in accordance with the following protocol:

100,000 T-cells were washed once with PBS, re-suspended in 50 μl PBS and fixated by adding 50 μl 4%-paraformaldehyde solution. The cells were then washed once in 200 μl PBS and then 2× in 200 μl Perm buffer (PBS with 2% FCS and 0.1% saponin. For staining with the Cbl-b antibody the cells were incubated with a 2 μg/ml solution in 50 μl Perm buffer for 30 minutes at 4°. The cells were then washed twice with Perm buffer and incubated for a further 30 minutes at 4° with directly labelled secondary antibody (anti mouse IgG-PE, manufacturer: Southern Biotech). Finally the cells were washed once with Perm buffer and once with FACS buffer and re-suspended in 250 μl FACS buffer for the FACS analysis.

In conformity with the western blot data a significant reduction in the Cbl-b staining intensity was also detected in the flow cytometry measurement (FIG. 3C). These data thus clearly prove that the protocol in accordance with the invention is suitable for the specific determination of the cellular Cbl-b protein content of human leukocytes by means of flow cytometry.

### Example 4

### The Combination of Cbl-b Detection with Further Immune Cell Markers Allows the Simultaneous Determination of the Cbl-b Protein Content is Various Disease-Relevant Immune Cells

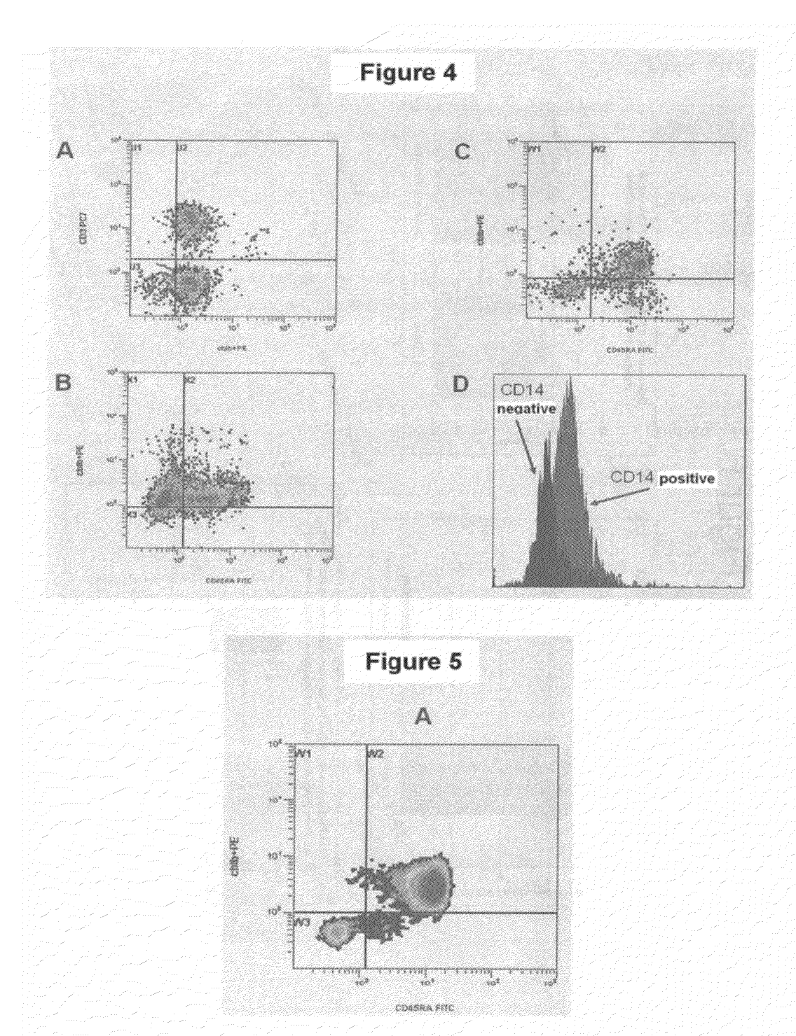

PBMCs from healthy volunteer donors were prepared in accordance with the standard protocol for density gradient centrifuging (Ficoll) and stained with Cbl-b antibody and secondary detection antibody as described above. The cells were also stained with antibodies directed against CD54A and CD3 (directly marked CD45RA-FITC and CD3-PE-Cy7 antibodies, manufacturer Invitrogen). The results of the FACS determination are set out in FIG. 4. By way of lateral (SSC) and forward (FSC) scattering determination individual cells types—if indicated—can be specifically determined. This shows that the Cbl-b content in the T-cell fraction of healthy persons is comparatively uniform (FIG. 4A, morphology gate, SSC and FSC adjustment to lymphocyte), irrespective of whether naïve (CD45RA+) or memory T-cells (CD45RA−) are involved (FIG. 4B, Cbl-b means fluorescence is almost identical 2.03 vs. 2.07). This also corresponds with the finding that only a minimal proportion of activated T-cells circulates in the blood of healthy persons. These data also show that in the CD3-negative fraction of the PBMCs the majority of the immune cells express Cbl-b FIG. 4C). The relevance of Cbl-b for the immune reactivity of B and invariant NKT cells was also shown in the literature (Kojo et al., PNAS (2009)/doi: 10.1073/pnas.0904078106).

A large proportion of the CD3-negative immune cells in PBMCs are however NK cells which are CD3-negative and CD45RA-positive. FIG. 4C shows that relevant quantities of Cbl-b are also expressed in these cells. FIG. 4D also shows that myeloid cells (morphology gate in SSC vs FSC on monocytes/macrophages) also express relevant quantities of Cbl-b proteins, whereby however preferably CD14-positive monocytes express Cbl-b protein in comparison with CD14-negative myeloid cells (predominantly macrophages).

### Example 5

### Expression of Cbl-b in NK-Cells

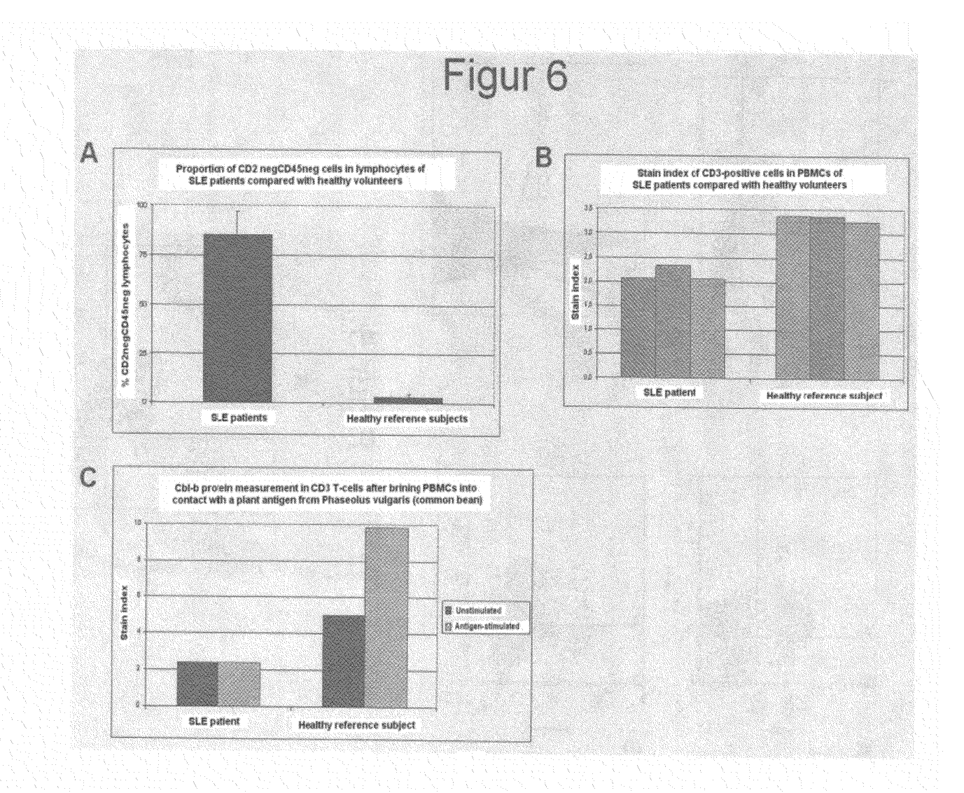

FIG. 5 shows the results of NK-cells isolated from the PBMCs by MACS (NK cell isolation kit, Invitrogen) and stained as in example 4 for the simultaneous determination of Cbl-b and CD45RA. It can be seen that all classic NK-cells (CD45RA-positive) express Cbl-b. The small proportion of CD45RA-negative cells in the preparation can in accordance with the literature be identified as “killer dentritic cells” which have properties of NK cells and dentritic cells (see for example Bonmort et al., Current Opinion in Immunology 2008, 20:558-565), as their cell morphology shows them to be slightly larger than classic lymphocytes, and can also be described through CD45RA-negative subsets (Bangert et al., J. Investigat. Dermatology 2003 121:1409-1418). Interestingly, precisely the Cbl-b-negative “killer dentritic cells” observed here have been identified as an important immune response factor to tumours (Larmonier et al., Cancer Immunol Immunother (2010) 59:1-11). Example 5 thus shows that the definition of distinct cellular sub-populations through the determination of their Cbl-b expression allows improved functional characterisation of the state of activation of the immune system within the context of tumorous diseases.

### Example 6

### Patients Suffering from an Autoimmune Disease Based on Pathologically Increased Immune Reactivity have a Reduced Content Cbl-b Content in T-Cells

A reduced Cbl-b protein content in immune cells leads to increased activation of the immune system. Whereas this is desirable in the case of a tumorous disease, pathologically increased immunity to endogenous antigens is pathologically relevant in the context of autoimmune diseases. The Cbl-b protein content of immune cells in patients with active systemic Lupus erythematosus (SLE) was therefore studied. PBMCs from SLE patients or healthy reference subjects were prepared and, as described in example 4, stained with antibodies to Cbl-b, CD45RA and CD3 and measured by means of flow cytometry. This allows the identification of various cell populations in terms of their Cbl-b protein content. Noticeably, in the SLE patients the proportion of CD3⁻CD45RA⁻ lymphocytes with low or no Cbl-b (below the FACS detection limit) was dramatically increased. (FIG. 7A, 3 donors per group, p=0.00025). In addition the Cbl-b protein content in CD3-positive T-cells was determined. FIG. 7B shows that the content of Cbl-b in T-cells of patients with autoimmune disease was considerably reduced in comparison with healthy reference subjects (stain index=median of the fluorescence of Cbl-b staining divided by that of the fluorescence of isotype staining p<0.0003). This is in conformity with the generally increased activation of immune cells in SLE patients (see for example Doreau et al., Nature Immunology 2009, doi:10.1038/ni.1741).

Allergies constitute a further pathological context of increased immune reactivities. PBMCs of an SLE patient and a healthy reference subject were thus brought into contact with harmless plant antigens (phytohaemagglutinine) from the common bean (Phaseolus vulgaris). In higher concentrations the antigen can lead to an activation of T-cells, which in the absence of other T-cell specific stimuli usually leads to an anergic reaction of the contacted T-cells. In accordance with this T-cells of a healthy reference subject reacted with a strong increase in the Cbl-b protein content (FIG. 7C) as is characteristic of anergic T-cells (incubation of 2 million PBMCs in 1 ml Xvivo Medium with 2 μl phytohaemaglutinine suspension (Invitrogen-GIBCO for 48 hours). In contrast to this the T-cells of an SLE patient, which already exhibit a reduced Cbl-b protein content, no longer reacted with an increase in the Cbl-b protein content.

Example 7 thus illustrates that the present method of determining the Cbl-b protein content in immune cells is particularly suitable in complex immune cell mixtures with various compositions and also allows predictions relating to the reaction of immune cells of patients to various stimuli on the basis of their Cbl-b content.

1.-27. (canceled)

28. A method of determining intracellular Cbl-b protein in cells of a sample comprising:
introducing an antibody, which binds Cbl-b intracellularly into a cell;
allowing contacting of the antibody and Cbl-b protein potentially present in the cell;
detecting binding events between the antibody and Cbl-b; and
quantifying the detected binding events;
wherein a content of Cbl-b protein is determined.

29. The method of claim 28, wherein the cells include leukocytes.

30. The method of claim 29, wherein the leukocyte is a peripheral blood mononuclear cell (PBMC).

31. The method of claim 28, wherein the cell comprises CD4+ lymphocytes, NK cells, monocytes, dendritic cells, and/or B cells.

32. The method of claim 28, wherein the antibodies are labelled.

33. The method of claim 28, wherein the cells are measured individually for detection of the binding events.

34. The method of claim 33, wherein the cell type is simultaneously classified or determined.

35. The method of claim 28, wherein the cells are measured with a high throughput of at least 20 cells per second.

36. The method of claim 28, wherein the cells are measured by flow cytometry.

37. The method of claim 28, wherein the proportion of cells in which Cbl-b is detected, and/or the quantity of Cbl-b protein in the cells is quantified.

38. The method of claim 28, wherein before detection of the binding events, the cells are simulated with an antigen.

39. The method of claim 38, wherein the cells are antigen presenting cells.

40. The method of claim 38, wherein the cells are simulated with the antigen before introduction of the antibody.

41. The method of claim 28, wherein the cells are treated with an immuno-stimulating substance or antibody to surface molecule.

42. The method of claim 41, wherein the immuno-stimulating substance is a cytokine or ligand of immunomodulating receptor.

43. The method of claim 42 wherein the immunomodulating receptor is a TLR (toll-like receptor).

44. The method of claim 41, wherein the antibody to surface molecule is a CD3 and/or CD28.

45. The method of claim 28, wherein the quantity of post-translationally modified Cbl-b protein is determined.

46. The method of claim 45, wherein the quantity of phosphorylated and/or ubiquitinated Cbl-b protein is determined.

47. A method of diagnosing a disease or predicting the occurrence or course of a disease comprising:
determining the Cbl-b protein content in cells of a sample of a subject by the method of claim 28;
comparing the Cbl-b protein content with diseased or healthy reference subjects; and
determining a difference between the Cbl-b protein content of the subject and the reference subject;
whereby a disease and/or the prognosis is/are determined.

48. The method of claim 47, wherein determining the Cbl-b protein content in cells of a sample is on at least 2 different days.

49. The method of claim 47, further defined as determining the Cbl-b protein at 2 different times and correlating of the Cbl-b protein content of the subject at the different times with the Cbl-b protein content of the reference subjects.

50. The method of claim 49, wherein the 2 or more times are at an interval of at least 2 days.

51. The method of claim 47, wherein the subject is a mammal.

52. The method of claim 51, wherein the mammal is a human.

53. The method of claim 47, further comprising detecting a proportion of cells in which Cbl-b is detected, and/or quantifying a quantity of Cbl-b protein in the cells and selecting an antigen which can trigger or influence the disease.

54. The method of claim 47, further defined as a method of determining a disease further defined as:
a chronic infection;
a tumor disease;
an inflammatory and/or autoimmune disease;
a disease comprising an immune response to an allotransplant; and/or
a disease comprising an immune reaction to allergens, exogenous antigens, or endogenous antigens.

55. A method of determining the immune reactivity of cells of a subject to an antigen, comprising:
bringing the cells into contact with the antigen;
determining the Cbl-b protein content in the cells of a sample of the subject by the method of claim 28;
comparing the Cbl-b protein content with reference values of a Cbl-b protein content in immune reactivity to a reference antigen, or absence of an immune reactivity to a reference antigen; and
determining a difference between the Cbl-b protein content of the subject and the reference values.

56. The method of claim 55, further defined as determining the immune reactivity of leukocytes to an antigen.

57. The method of claim 55, wherein the reference values have been determined from samples of other subjects.

58. The method of claim 57, wherein the reference values have been determined from samples of other subjects with the antigen being defined as identical to the reference antigen.

59. The method of claim 55, wherein the antigens are allergens, exogenous antigens or endogenous antigens of the subject.

60. The method of claim 55, wherein a content or activity of PKCtheta is also determined and used to determine the immune reactivity of the cells.

61. The method of claim 55, wherein the content or activity of PKCtheta is compared with a PKCtheta reference value.

62. A method of intracellular determination of Cbl-b comprising:
obtaining an antibody which binds an epitope of Cbl-b in the intracellular environment; and
determining intracellular Cbl-b using the antibody.

63. The method of claim 62, wherein the antibody binds an epitope of the C-terminal 300 amino acids of Cbl-b.

64. The method of claim 62, further defined as comprising determining intracellular Cbl-b protein in cells of a sample by a method comprising:
introducing an antibody which binds Cbl-b intracellularly into a cell;
allowing contacting of the antibody and Cbl-b protein potentially present in the cell;
detecting binding events between the antibody and Cbl-b; and
quantifying the detected binding events.

65. The method of claim 64, further defined as a method of determining a disease further defined as:
a chronic infection;
a tumor disease;
an inflammatory and/or autoimmune disease;
a disease comprising an immune response to an allotransplant; and/or
a disease comprising an immune reaction to allergens, exogenous antigens, or endogenous antigens.

66. A kit for practicing the method of claim 28, which comprises an antibody which binds an epitope of Cbl-b in the intracellular environment.
